(19) United States
(12) Patent Application Publication
(10) Pub. No.: US 2002/0051991 A1
(43) Pub. Date: May 2, 2002

(54) Rapid generation of diverse nucleoside and nucleotide libraries

(76) Inventors: Zhi Hong, Aliso Viejo, CA (US); Haoyun H. An, Carlsbad, CA (US); Frank Rong, Costa Mesa, CA (US)

Correspondence Address: RUTAN & TUCKER, LLP, P.O. BOX 1950, COSTA MESA, CA, 92628-1950, US

(21) Appl. No.: 09/904,248

(22) Filed: Jul. 11, 2001

Related U.S. Application Data
(60) Provisional application No. 60/217,635, filed on Jul. 11, 2000.

(51) Int. Cl. C12Q 1/68; C07H 19/00; G01N 33/53

(52) U.S. Cl. 435/6; 435/7.1; 536/27.13

(57) ABSTRACT

Methods for generation of nucleoside libraries are provided in which nucleosides or nucleoside precursors (e.g., a sugar or a base) are coupled to a solid phase and sequentially reacted with various substrates.

This application claims the benefit of U.S. provisional application No. 60/217,635 filed Jul. 11, 2000, which is incorporated herein by reference.

### FIELD OF THE INVENTION

The invention is directed to methods for synthesizing large collections of diverse nucleosides.

### BACKGROUND OF THE INVENTION

Nucleosides and related compounds interact with many biological targets, and some nucleoside analogues have been used as antimetabolites for treatment of cancers and viral infections. After entry into the cell, many nucleoside analogues can be phosphorylated to monophosphates by nucleoside kinases, and then further phosphorylated by nucleoside monophosphate kinases and nucleoside diphosphate kinases to give nucleoside triphosphates. Once a nucleoside analogue is converted to its triphosphate inside the cell, it can be incorporated into DNA or RNA. Incorporation of certain unnatural nucleoside analogues into nucleic acid replicates or transcripts can interrupt gene expression by early chain termination, or by interfering with function of the modified nucleic acids. In addition, certain nucleoside analogue triphosphates are a very potent, competitive inhibitor of DNA or RNA polymerases, which can significantly reduce the rate at which the natural nucleoside can be incorporated. Many anti-HIV nucleoside analogues fall into this category, including 3′-C-azido-3′-deoxythymidine, 2′,3′-dideoxycytidine, 2′,3′-dideoxyinosine, and 2′,3′-didehydro-2′,3′-dideoxythymidine. The nucleoside analogues can also act in other ways, for example, causing apoptosis of cancer cells and modulating immune systems. In addition to the nucleoside antimetabolites, a number of nucleoside analogues that show very potent anticancer and antiviral activities act through other mechanisms. Some well-known nucleoside anticancer drugs are thymidylate synthase inhibitors such as 5-fluorouridine, and adenosine deaminase inhibitors such as 2-chloroadenosine. A well-studied anticancer compound, neplanocin A, is an inhibitor of S-adenosylhomocysteine hydrolase, which shows potent anticancer and antiviral activities.

Many of these nucleoside analogues that can inhibit tumor growth or viral infections are also toxic to normal mammalian cells, primarily because these nucleoside analogues lack adequate selectivity between the normal cells and the virus-infected host cells or cancer cells. For this reason many otherwise promising nucleoside analogues fail to be human therapeutics. Selective inhibition of cancer cells or host cells infected by viruses has been an important subject for some time, and tremendous efforts have been made to search for more selective nucleoside analogues. In general, however, a large pool of nucleoside analogues is thought to be necessary in order to identify highly selective nucleoside analogues. Unfortunately, the classical method of synthesizing nucleosides and nucleotides having desired physiochemical properties, and then screening them individually, takes a significant amount of time to identify a lead molecule. Although thousands of nucleoside analogues were synthesized over the past decades, if both sugar and base modifications are considered, many additional analogues are still waiting to be synthesized.

During the last few years combinatorial chemistry has been used to generate huge numbers of organic compounds, resulting in large compound libraries. If nucleosides could be made through a combinatorial chemistry approach, a large number of nucleoside analogues could be synthesized within months instead of decades, and large nucleoside libraries could be developed.

A combinatorial chemistry approach to nucleosides may also encourage a focus beyond previously addressed biological targets. For example, in the past nucleoside analogues were usually designed as potential inhibitors of DNA or RNA polymerases and a handful of other enzymes and receptors such as inosine monophosphate dehydrogenase, protein kinases, and adenosine receptors. If a vast number of diversified nucleoside analogues could be created, their use may be far beyond these previously recognized biological targets, which would open a new era for the use of nucleoside analogues as human therapeutics.

Although a combinatorial chemistry approach has been proven to work well with many types of compounds, there are certain hurdles to the generation of nucleoside libraries. Most nucleoside analogues contain a sugar moiety and a nucleoside base, which are linked together through a glycosidic bond. The formation of the glycosidic bond can be achieved through a few types of condensation reactions. However, most of the reactions do not give a good yield of desired products, which may not be suitable to generation of nucleoside libraries. In addition, the glycosidic bonds in many nucleosides are in labile to acidic condition, and many useful reactions in combinatorial chemistry approaches cannot be used in the generation of nucleoside analogue libraries.

The generation of combinatorial libraries of chemical compounds by employing solid phase synthesis is well known in the art. For example, Geysen, et al. (Proc. Natl. Acac. Sci. USA, 3998 (1984)) describes the construction of a multi-amino acid peptide library; Houghton, et al. (Nature, 354, 84 (1991)) describes the generation and use of synthetic peptide combinatorial libraries for basic research and drug discovery; Lam, et al. (Nature, 354, 82 (1991)) describes a method of synthesis of linear peptides on a solid support such as polystyrene or polyacrylamide resin. However, no methods have been disclosed for generating libraries of nucleosides and nucleotides using solid phase synthesis. Therefore, there is still a need to provide methods for generation of nucleoside and nucleotide libraries.

### SUMMARY OF THE INVENTION

The present invention is directed to method of generating nucleoside libraries in which nucleosides or nucleoside precursors are coupled to a solid phase and sequentially reacted with various substrates.

In one aspect of the inventive subject matter, a method comprises a step in which a first and second nucleoside are provided, wherein each of the nucleosides has a first reactive group protected with a first protecting group and a second reactive group protected with a second protecting group, and wherein the first and second nucleosides are coupled to a solid support. In a further step, the first protecting group is removed from the first and second nucleosides and the first reactive group of the first nucleoside is reacted with a first reagent while the first reactive group of the second nucleoside is reacted with a second reagent. In a still further step, the second protection group is removed from the first and second nucleosides and the second reactive group of the first nucleoside is reacted with a third reagent and the second reactive group of the second nucleoside is reacted with a fourth reagent, thereby creating a nucleoside library with at least two library members.

In another aspect of the inventive subject matter, the first and second reagents are chemically identical, and it is further contemplated that the step of reacting the second reactive group of the first nucleoside with the third reagent and the second reactive group of the second nucleoside with the fourth reagent is performed in separate compartments. It is also contemplated that at least one of the first and second nucleosides is coupled to a solid support via a linker molecule, preferably via an acetal bond. With respect to the nucleosides it is contemplated that the nucleosides may be chemically distinct or identical, and that at least one of the first and second nucleosides comprises a ribofaranose and a natural nucleoside base. Preferred solid supports comprise a polystyrene resin or a polystyrene-polyethylene glycol copolymer, and preferred first protecting groups comprise a p-methoxy-benzyl group and second protecting groups comprise a benzyl group.

In another aspect of the inventive subject matter, a method comprises a step in which a first sugar and a second sugar are provided, wherein each of the first and second sugars have at least one reactive group and are covalently bound to a solid support. In a further step the first and second reactive groups are reacted with a first heterocyclic base and a second heterocyclic base, respectively, thereby forming a first nucleoside and a second nucleoside. In another step the first and second nucleosides are reacted with a first and second reagent in at least one subsequent reaction, respectively, thereby forming a first modified nucleoside and a second modified nucleoside, and thereby creating a nucleoside library with at least two library members.

In a still further aspect of the inventive subject matter, first and second sugars are chemically identical, and it is preferred that the step of reacting the first and second nucleosides with a first and second reagent, respectively, is performed in separate compartments. It is still further preferred that the sugar is coupled to a solid support via a linker molecule, most preferably via an acetal bond. Heterocyclic bases may be chemically distinct from each other and may advantageously comprise an imidazole, a thiazole, or an oxazole.

Various objects, features, aspects and advantages of the present invention will become more apparent from the following detailed description of preferred embodiments of the invention.

### DETAILED DESCRIPTION OF THE INVENTION

The terms “combinatorial synthesis strategy” or “combinatorial chemistry” generally refer to an ordered strategy for the parallel synthesis of diverse compounds by sequential addition of reagents, which leads to the generation of large chemical libraries. Thus, combinatorial chemistry refers to the systematic and repetitive, covalent connection of a set of different building blocks of varying structures to each other to yield large arrays of diverse molecular entities.

The term “nucleoside library” as used herein refers to a plurality of chemically distinct nucleosides, wherein at least some of the nucleosides have been synthesized from a common synthesis intermediate. The term “synthesis intermediate” explicitly excludes starting materials of the synthesis. It is generally contemplated that the complexity of contemplated libraries is at least 20 distinct nucleosides, more typically at least 100 distinct nucleosides, and most typically at least 1000 distinct nucleosides. The term “library compound” as used herein refers to a nucleoside within the nucleoside library.

As also used herein, the term “heterocyclic base” refers to any compound in which a plurality of atoms form a ring via a plurality of covalent bonds and that further includes at least one atom other than a carbon atom. Particularly contemplated heterocyclic bases include purine bases, pyrimidine bases, 5- and 6-membered rings with nitrogen as the non-carbon atom (e.g., imidazole, pyrrole, triazole, dihydropyrimidine), and a 5-membered ring fused to a 6-membered ring (e.g., purine, pyrrolo[2,3-d]pyrimidine), and a 6-membered ring fused to another 6-membered or higher ring (e.g., pyrido[4,5-d]pyrimidine, benzodiazepine). Examples of these and further preferred heterocyclic bases are given below.

The term “nucleoside” refers to all compounds in which a heterocyclic base is covalently coupled to a sugar, and an especially preferred coupling of the nucleoside to the sugar includes a C1′-glycosidic bond of a carbon atom in a sugar to a carbon- or heteroatom (typically nitrogen) in the heterocyclic base. The term “nucleoside analog” as used herein refers to all nucleosides in which the sugar is not a ribofuranose and/or in which the heterocyclic base is not a naturally occurring base (e.g.,A, G, C, T, I, etc.).

As further used herein, the term “linker” refers to a molecule or group of molecules attached to a solid support and spacing a synthesized compound from the solid support. The term “solid support” or “support” refers to a material or group of materials that are either insoluble, and/or that have a rigid or semi-rigid surface or surfaces. In many embodiments, at least one surface of the solid support will be substantially flat, although in some alternative embodiments it may be desirable to physically separate synthesis regions for different compounds with, for example, wells, raised regions, pins, etched trenches, or the like. According to other embodiments, the solid support(s) will take the form of insoluble polymers, wells, beads, resins, gels, microspheres, or other geometric configurations.

In a particularly preferred aspect of the inventive subject matter, a nucleoside library is generated in a reaction sequence as outlined below in which a plurality of nucleosides are coupled to a solid support and modified to include various substituents. Here, a commercially available nucleoside (e.g., adenosine or cytosine; heterocyclic base is represented by B) is converted to 11 by silylation reaction using standard reaction conditions well-known in the art. The C2′-hydroxyl of 11 is then protected with p-methoxybenzyl (MPM), followed by removal of the silyl-protecting group, to give 13. The protection of the C5′-hydroxyl with dimethoxytrityl (DMT) and the C3′-hydroxyl with benzoyl gives compound 15. Exemplary suitable reaction protocols and procedures for protection and deprotection of the hydroxyl groups in the sugar portion of contemplated nucleosides are well known in the art and are described in “Protective Groups in Organic Synthesis” by Peter G. M. Wuts, Theodora W. Greene (John Wiley & Sons; ISBN: 0471160199). After removal of DMT using standard procedures (supra), the 5′-hydroxy of 16 is linked to the commercially available solid support 17 through an acetal bond, which can be cleaved at a mild acidic condition. The linking reaction of the nucleoside to the solid support may be performed using various routes, and exemplary suitable linking reactions to the solid phase are described in “Organic Synthesis on Solid Phase—Supports, Linkers, Reactions” by Florencio Zaragoza Dorwald et al. John (Wiley & Sons; ISBN: 3527299505), or in “Solid-Phase Synthesis and Combinatorial Technologies” by Pierfausto Seneci (John Wiley & Sons; ISBN: 0471331953). The resulting compound 18 is subjected to a basic hydrolysis and a subsequent oxidation to give keto compound 20, which can be used as a starting material in the synthesis of a nucleoside analogue library.

[chemical structure]

Compound 20, which is attached to the solid support, can then be reacted with a variety of nucleophiles including Grignard reagents, organolithium reagents and Wittig reagents as depicted below. It should further be appreciated that nucleophiles can attack the keto group from both the α and β-faces of the nucleoside to yield an even larger number of derivatives. The resulting compound 21 is subjected to removal of MPM with DDQ or certain Lewis acids to give compound 22. Oxidation of compound 22 will give the 2′-keto compound 24, which can be subjected to the same set of reactions as the 3′-keto compound 20. Since both 2′- and 3′-keto compounds 20 and 24 can generate both α- and β-substituted products, a library of hundreds of sugar-modified nucleosides can be readily produced. The resulting compound 25 attached to the solid supports can be treated with weak acid such as 80% acetic acid to release the modified nucleoside 26 from the solid supports. It is further generally contemplated that contemplated nucleosides can be synthesized on a multiple-vessel synthesizer and can be obtained as a mixture of 1-4 compounds from one reaction vessel.

[chemical structure]

The so generated nucleoside library may then be subjected to direct testing (i.e., without cleavage of the nucleosides from the solid phase) for nucleosides with desired properties, or in alternative aspects the nucleosides may be cleaved (supra) from the solid support prior to testing.

[chemical structure]

In alternative aspects of the inventive subject matter, it should be appreciated that numerous nucleosides other than adenosine or cytosine may also be employed, and particularly contemplated nucleosides may comprise a sugar other than a ribofuranosyl and/or a heterocyclic base other than a naturally occurring base (i.e., adenine, guanine, cytidine, thymine, and uracil).

For example, suitable nucleosides may include various heterocyclic bases, and particularly contemplated heterocyclic bases include substituted and unsubstituted purines and pyrimidines, and substituted and unsubstituted triazoles, pyrrolo[2,3-d]pyrimidines, and pyrido[4,5-d]pyrimidines. The term “substituted” molecule as used herein means that the molecule further comprises a functional group, and particularly contemplated functional groups include nucleophilic (e.g., —NH₂, —OH, —SH, —NC, etc.) and electrophilic groups (e.g., C(O)OR, C(X)OH, etc.), polar groups (e.g., —OH), ionic groups (e.g., —NH₃⁺), halogens (e.g., —F, —Cl), and all chemically reasonable combinations thereof. There are numerous heterocyclic bases known in the art, all of which are contemplated suitable for use herein, and an exemplary collection and synthetic procedures for such heterocyclic bases are described in Handbook of Nucleoside Synthesis by Helmut Vorbruggen, Carmen Ruh-Pohlenz; John Wiley & Sons; ISBN: 0471093831.

In further examples, the sugar moiety of contemplated nucleosides need not be restricted to a ribofuranose, and numerous alternative sugars are also contemplated suitable for use in conjunction with the teachings herein. As used herein, the terms “sugar” and “sugar moiety” refer to all carbohydrates and derivatives thereof, wherein particularly contemplated derivatives include sugars in which a chemical group has been deleted, substituted, or added. Among other things, especially contemplated deletions include 2′-deoxy and/or 3′-deoxy sugars. Especially contemplated substitutions include replacement of the ring-oxygen with sulfur or methylene, or replacement of a hydroxyl group with a halogen, an amino-, sulfhydryl-, or methyl group, and especially contemplated additions include methylene phosphonate groups. Moreover, it should be appreciated that contemplated sugars may have D-configuration as well as L-configuration, and it should still further be recognized that suitable sugars also include open-chain sugars, and conformationally restrained or locked sugars. Exemplary alternative sugars particularly contemplated herein are described by Ichikawa et al. in Curr Med Chem 2001 Mar;8(4):385-423.

It should still further be appreciated that the sugar may be coupled to the solid phase in a position other than the C5′-position, and alternative positions especially include the C2′- and C3′-position. Consequently, it is contemplated that the C5′-position (or homologous position) of contemplated sugars may also be modified, and particularly contemplated modifications include phosphorylation (especially including mono-, di-, and triphosphorylation), and addition of affinity groups (e.g., biotin, oligo-his) and/or labeling groups (e.g., fluorophore, radioisotope, etc.).

While it is generally preferred that the modification of the nucleoside includes deprotection of an OH group in the sugar moiety of the nucleoside, oxidation of the OH group to form a carbonyl atom, and subsequent nucleophilic attack of the carbonyl atom, numerous alternative modification reactions (including modification of the sugar moiety and/or modification of the heterocyclic base moiety) are also contemplated. For example, the deprotected OH group may act as a nucleophile with an electrophilic center of a substrate. Alternatively, where the OH group was previously replaced with a NH2 group, the NH₂ group may be similarly modified. Consequently, first and second reactive groups may include all groups that are covalently bound to the sugar and/or heterocyclic moiety and that may participate in a subsequent modification reaction. However, especially contemplated reactive groups include carbonyl atoms, —OH, ═O, NH₂, and —SH.

Consequently, it should be appreciated that numerous protection groups other than p-methoxybenzyl, benzyl, and dimethoxytrityl are also considered appropriate, and the choice of a particular protecting group will typically depend on the chemical nature of the reactive group. There are numerous protecting groups known in the art, and all of these are considered suitable for use in conjunction with the teachings herein. An exemplary collection of suitable protecting groups and their reactions/reaction conditions with various reactive groups is described in “Protective Groups in Organic Synthesis” (supra).

It should further be appreciated that the chemical nature of appropriate reagents will vary considerably, and predominantly depend on the chemical nature of the reactive group(s) and the type of desired modification. However, it is especially contemplated that at least one of the first through fourth reagents includes nucleophilic reagent, including a Grignard reagent, an organolithium reagent, or a Wittig reagent. Thus, especially preferred reagents include R-MgBr, wherein R is substituted or unsubstituted alkyl, substituted or unsubstituted alkenyl, substituted or unsubstituted alkynyl, substituted or unsubstituted aryl, or substituted or unsubstituted alkaryl. Alternatively, suitable reagents include nucleophiles, electrophiles, acids, and bases.

The terms “alkyl” and “unsubstituted alkyl” are used interchangeably herein and refer to any linear, branched, or cyclic hydrocarbon in which all carbon-carbon bonds are single bonds. The terms “alkenyl” and “unsubstituted alkenyl” are used interchangeably herein and refer to any linear, branched, or cyclic alkyl with at least one carbon-carbon double bond. The term “substituted alkenyl” as used herein refers to any alkenyl that further comprises a functional group, and particularly contemplated functional groups include those discussed above. Furthermore, the terms “alkynyl” and “unsubstituted alkynyl” are used interchangeably herein and refer to any linear, branched, or cyclic alkyl or alkenyl with at least one carbon-carbon triple bond. The term “substituted alkynyl” as used herein refers to any alkynyl that further comprises a functional group, and particularly contemplated functional groups include those discussed above. The terms “aryl” and “unsubstituted aryl” are used interchangeably herein and refer to any aromatic cyclic alkenyl or alkynyl. The term “substituted aryl” as used herein refers to any aryl that further comprises a functional group, and particularly contemplated functional groups include those discussed above. The term “alkaryl” is employed where the aryl is further covalently bound to an alkyl, alkenyl, or alkynyl.

With respect to the solid phase it is contemplated that suitable solid phases may be of any appropriate shape, size and composition. For example, preferred support include Merrifield resin (Chloromethyl polystyrene—available from Sigma-Aldrich, St. Louis, Mo.), ArgoGel (available from Argonaut, San Francisco, Calif.), Sasrin resin (a polystyrene resin available from Bachem Bioscience, Switzerland), TentaGel S AC, TentaGel PHB, or TentaGel S NH2 resin (polystyrene-polyethylene glycol copolymer resins available from Rappe Polymere, Tubingen, Germany). Other preferred solid phases include glass, as described in U.S. Pat. No. 5,143,854.

It is further generally preferred that the bead size is in the range of less than 1 μm to 100 μm, but a more massive solid support of up to 1 mm in size may sometimes be used. Other supports are commercially available and described by Novabiochem, La Jolla, Calif.

Another class of preferred solid support comprises a “soluble” polymer support. Typically, any of polyethylene glycol, polyvinylalcohol, polyvinylalcohol co-polymerized with polyvinyl pyrrolidine or derivatives thereof is used as the soluble support. See Janda and Hyunsoo (1996) Methods Enzymol. 267:234-247; Gravert and Janda (1997) Chemical Reviews 97:489-509; and Janda and Hyunsoo, PCT publication No. WO 96/03418.

With respect to the reactions and reaction sequence, it should be appreciated that all chemically reasonable combinations are contemplated. For example, where the first and second reagent are chemically identical, the first and second nucleoside may be reacted in a single compartment. Thus, diversity will be introduced into the library only in a subsequent reaction (provided the first and second nucleoside are chemically identical). Alternatively, where the first and second reagents are chemically distinct (i.e., chemically not identical) the first and second nucleoside may be reacted in separate compartments, thereby introducing diversity, which may be rendered more complex in a subsequent reaction. However, it is also contemplated that where the first and second reagents are chemically distinct (i.e., chemically not identical) the first and second nucleoside may be reacted in a single compartment. The same considerations apply for subsequent reactions of the second reactive group of the first nucleoside with a third reagent and the second reactive group of the second nucleoside with a fourth reagent.

Where coupling of the nucleoside includes a linker between the nucleoside and the solid phase, it is especially preferred that such coupling includes an acetal bond, and that the linker comprises a dihydropyran portion. However, it should be appreciated that numerous alternative couplings are also suitable, and will at least in part depend on the type of solid phase employed. Especially preferred linkers are hydrolyzable under mild acidic conditions. Thus particularly suitable linkers include silyl chloride (Randolph, et al. (1995) J. Am. Chem. Soc. 117, 5712); succinic anhydride (The Combinatorial Index, (1998), 60); tetrahydro-pyranyl (Thompson, et al. (1994), Tetrahedron Letts. 35, 9333); and sulfonate ester (Hunt, et al. (1996), J. Am. Chem. Soc. 118, 9998). Moreover, it is contemplated that preferred linker molecules will have lengths sufficient to allow the compounds to which they are bound to interact freely with any molecules exposed to the solid support surface, such as synthetic reagents or receptors, which are an object of study.

In another particularly preferred aspect of the inventive subject matter, a nucleoside library is generated in a reaction sequence as outlined below, in which a protected sugar is coupled to a solid support and then covalently bound to a heterocyclic base to form a nucleoside. The nucleoside is then modified in a modification reaction to form a modified nucleoside.

Here, a protected sugar is coupled to a solid phase in a procedure similar to the coupling procedure as described above to yield compound 27, which is then reacted with 3-cyano-1,2,4-triazole to generate nucleoside analog 28. The cyano group in nucleoside analog 28 is hydrolyzed under various conditions (e.g., H₂S and base, or aqueous NH₃ and base) to generate modified nucleoside 29.

[chemical structure]

With respect to the solid phase, the protecting groups and coupling/decoupling conditions of solid phase and protecting the sugar the same considerations as described above apply. In further alternative aspects the sugar need not be restricted to a protected ribofuranose, and it should be appreciated that a particular chemical nature of the sugar is not limiting to the inventive subject matter. Therefore, all previously contemplated sugars are considered suitable for use in conjunction with the teachings presented herein, however, particularly contemplated sugars include modified and unmodified ribofuranoses such as 3′-deoxyribofuranose, 2′,3′-dideoxyribofuranose, 3′-fluoro-3′-deoxyribofuranose, 3′-azido-3′-deoxyribofuranose, etc.

Consequently, it is contemplated that the reactive group in contemplated sugars may vary considerably, and especially preferred reactive groups in such sugars include carbonyl atoms, —OH, —CN, ═O, and —NH₂. However, it is generally contemplated that all reactive groups are suitable so long as such groups are capable of formation of a covalent bond with a heterocyclic base, and contemplated sugars may further comprise more than one reactive group. Moreover, it is contemplated that the reactive group in suitable sugars may be in a position other than the C1′-position, and particularly contemplated alternative positions include the C2′- and C3′-positions. Thus, it should be appreciated that appropriate sugars may further include a C′1-glycosidically bound heterocyclic base.

Similarly, the heterocyclic base need not be restricted to 3-cyano-1,2,4-triazole, and all of the previously contemplated heterocyclic bases are considered suitable for use herein. However, especially preferred heterocyclic bases include an activated group that can react under conditions that preserve the protecting groups on the sugar and the bond of the sugar to the solid phase.

In a further particularly preferred aspect of the inventive subject matter, a nucleoside library is generated in a reaction sequence as outlined below, in which a nucleoside is protected, coupled to a solid support, and then modified on the base portion in a modification reaction to form a modified nucleoside.

[chemical structure]

Here, a commercially available nucleoside (2′-O-methyluridine) 30 is reacted with a solid phase and a protecting group to yield the protected nucleoside 31. 31 is subsequently reacted with tris(isopropyl)benzenesulfonyl chloride to form compound 32, which serves as a substrate in a parallel array synthesis of nucleoside library 33. The library members are then deprotected and cleaved from the solid support to form nucleoside library 34.

With respect to the solid phase, the protecting groups and coupling/decoupling conditions of solid phase and protecting the sugar the same considerations as described above apply. In further alternative aspects the nucleoside need not be restricted to 2′-O-methyluridine, and it should be appreciated that the particular chemical nature of the nucleoside is not limiting to the inventive subject matter. Therefore, all previously contemplated nucleosides, and especially modified nucleosides (which may further be in form of a library) are considered suitable for use in conjunction with the teachings presented herein. However, particularly contemplated nucleosides include modified and unmodified ribofuranose as a sugar portion (e.g., 3′-deoxyribofuranose, 2′,3′-dideoxyribofuranose, 3′-fluoro-3′-deoxyribofuranose, 3′-azido-3′-deoxyribofuranose, 2′-beta-alkyl/alkenyl/alkynyl/halo/polyhaloalkyl ribofuranose, etc).

Furthermore, it is contemplated that the reactive group in contemplated nucleosides may vary considerably, and especially preferred reactive groups in contemplated nucleosides include carbonyl carbon atoms, —OH, —CN, ═O, and —NH₂ and activated derivatives thereof. Particularly preferred activated derivatives include covalent modifications of contemplated reactive groups with a leaving group (e.g., TPS, Mesyl, Tosyl, halo etc.). However, it is generally contemplated that all reactive groups and their derivatives are suitable so long as such groups are capable of the formation of a covalent bond with a modifying reagent (e.g., a nucleophile, an electrophile, an acid, or a base), and contemplated nucleosides may further comprise more than one reactive group. Still further, it is contemplated that the reactive group in suitable nucleosides need not be limited to a particular position in the base or sugar moiety. Consequently, suitable reactive groups may be in any position (including heteroatoms and substituents on the base) of the nucleoside.

For example, where the nucleoside has two reactive groups on the heterocyclic base portion, a library may be constructed following a protocol as depicted below in which a nucleoside is reacted on the base portion with a first set of reagents, protected, and coupled to a solid support. The so generated nucleosides are then modified on the base portion in a further modification reaction with a second set of reagents.

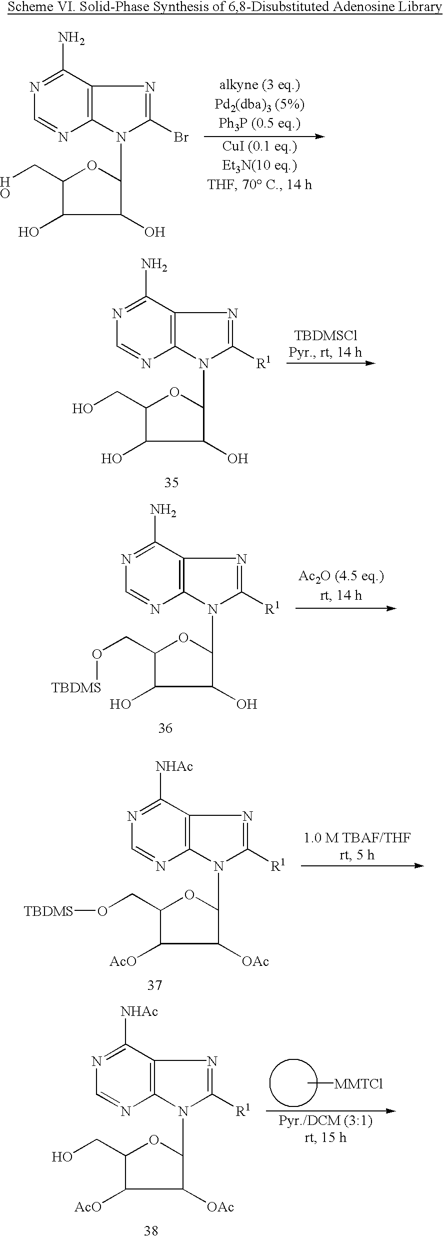

While it is generally preferred to employ a purine nucleoside for such libraries, it should be appreciated that in alternative aspects numerous nucleosides other than purine nucleosides are appropriate. For example, suitable nucleosides include pyrimidine nucleosides, nucleosides with a modified and/or unmodified sugar portion, and nucleosides with contemplated heterocyclic bases. With respect to the reactive group and reagents, the same consideration as discussed above apply.

Thus, it is generally contemplated that a method of generating a nucleoside library may comprise one step in which a first nucleoside and a second nucleoside are provided, wherein each of the nucleosides has a reactive group and is coupled to a solid support. In a further step, the reactive group of the first and second nucleosides is reacted with a first reagent and second reagent, respectively, thereby forming a first modified nucleoside and a second modified nucleoside, wherein the second modified nucleoside is chemically distinct from the first modified nucleoside.

In especially contemplated aspects, the reactive group is disposed in the heterocyclic base or the sugar moiety of the nucleoside, and where contemplated nucleosides include more than one reactive group it is contemplated that at least one of the reactive groups is disposed in the heterocyclic base, and the other reactive group(s) is disposed in the sugar moiety. With respect to suitable sugar and heterocyclic base moieties, reactive groups, and reagents the same considerations as discussed above apply. However, it is particularly preferred that at least one of the first and second nucleosides comprises a purine heterocyclic base with at least one, more preferably two reactive groups. Where contemplated nucleosides comprise at least two reactive groups, it is contemplated that suitable methods further comprise a step of reacting the second reactive group of the first and second nucleoside with a third reagent and a fourth reagent, respectively.

### EXAMPLES

The following protocols are provided to illustrate exemplary reaction conditions and substrates for libraries according to the inventive subject matter. However, it should be recognized that the examples may be modified to a large extent without departing from the inventive concepts presented herein. For example, one or more of the solid support, sugar and heterocyclic base may be replaced to generate a library with different library members. Similarly, the reaction sequence or substrates/reagents may be modified to generate different complexity in contemplated libraries.

### 2′-O-methyl-N4-Substituted Cytidine Libraries

A solution of commercially available 2′-O-methyluridine (30, supra) (2.17 g) in 25 ml of pyridine was added to a shaker funnel containing 4.05 g of 4-methyoxytrityl chloride resin (Novabiochem, loading capacity, 1.73 mmol/g). 4-N,N-Dimethylaminopyridine (DMAP) was added, and the reaction mixture was shaken at room temperature for 2 days. The mixture was filtered and the resin was washed 4 times with pyridine-DMF (1:1) and 4 times with dichlorormethane. The resulting uridine-substituted resin was swelled in 20 ml of pyridine, 10 ml of dichloromethane and 3.0 ml of triethylamine. T-Butyldimethylsilychloride (5.27 g, 5 eq.) and imidazole (2.38 g, 5 eq) were added to the mixture followed by 5 ml of DMF to improve the solubility. The mixture was shaken at room temperature for 24 hours and filtered. The resin was washed 4 times with pyridine-DMF (1:1) and 3 times with dichloromethane, and dried under vacuum to provide dried resin 31 loaded with protected cytidine.

A mixture of resin 31, DMAP (100 mg), dichloromethane (30 ml) and triethylamine (6.8 ml) was shaken at room temperature for 30 minutes. 2,4,6-tris(isopropyl)benzene-sulfonyl chloride (TIP-Cl, 4.24 g, 2 eq) was added. The resulting mixture was shaken at room temperature for 24 hours. 2 ml of methanol was added to consume the excess amount of TIP-Cl, shaken and filtered. The resin was washed 5 times with pyridine-DMF (1:1) and 3 times with dichloromethane, and dried under vacuum to provide 7.2 g of resin 32, which is confirmed by MAS NMR spectrometry and ready for the parallel array synthesis of nucleoside library 34.

50 mg of resin 32 was added to each of the 96 wells on an ACT parallel synthesizer. 1 ml of base (0.3 M DMAP in pyridine containing diisopropylethylamine) and 0.65 ml of each of 96 amines (1 M in DMF) were added to each of the 96 reaction vessels. The sealed reaction vessels in the reaction block were shaken at room temperature for 6 hours. The solvent was filtered off by vacuum. The resins were washed 3 times with DMF, 3 times with DCM-MeOH, and 3 times with dichloromethane to give a library of 96 resins 33.

1 ml of DMF and 1 ml of tetrabutylammonium fluoride in THF (1 M) were added to each of the 96 reaction vessels. The reaction block was shaken at room temperature for 5 hours, filtered and washed 3 times with DMF, 3 times with 40% water in methanol, and 3 times with dichloromethane. To the reaction 96 vessels containing resins were added 1.5 ml of 2% trifluoroacetic acid solution in dichloroethane. After shaking for 2 minutes, the filtrates were collected to 96 different vials. The resins were further washed with methanol and the filtrates were combined to the corresponding 96 vials. The solutions of the 96 samples were dried to provide 96 nucleosides 34 in 20-30 mg. The samples were analyzed by TLC and LC-MS spectrometry. LC-MS analysis of these samples confirmed the integrity and purity. Sample purity of the samples ranges from 70-100%.

The following R-NH₂ reagents (building blocks) were used for this library: 1-(benzyl)benzylamine, 2-phenyl-n-propylamine, m-trifluorobenzylamine, 2,2-diphenylethylamine, cyclobutylamine, metylcyclohexylamine, 2-methylpropylamine, allylcyclopentanylamine, N-methyl-4-piperidinylmethylamine, 4-hydroxypiperidine, N4-benzylpiperazine, p-methoxybenzylamine, 2-N,N-dimethylethylamine, N,N-bis(isopripyl)ethylamine, Piperazine, 2-ethylhexylamine, 5-methyl-2-fiiranosylmethylamine, N,N-dimethylpropylamine, 3-(N,N-dimethylamino)-2,2-dimethylpropylamine, 2-methylbutylamine, o-ethoxybenzylamine, 3-(2-methyl-N-piperidinylpropylamine, 2-pyrrolidinylethylamine, 4-N-methylpiperazine, 2-morpholinylethylamine, N4-hydroxyethylpiperazine, N-methylethylenediamine, 3-morpholinylpropylamine, pyridinyl-2-ethylamine, butylamine, hexylamine, methylamine, 2-hydroxyethylamine, 2-(N,N-dimethylamino)ethylamine, 3-methoxypropylamine, 2-methoxylethylamine, ethylamine, 2-isopropylamine, methylethylamine, 2-methylthioethylamine, di-n-butylamine, dimethylamine, allylamine, cyclopantylamine, 2-(N-methyl-pyrrolidin-2-yl)ethylamine, tetrahydrofuranosyl-2-methylamine, piperidine, N-benzyl-4-aminopiperidine, cyclopropylmethylamine, cyclopropylamine, 3-methylpiperizine, 4-piperidin-1-ylpiperidine, cyclohexylamine, piperazine, 4-pyridin-2-ylpiperazine, N-methylpiperazine, N-(2-methoxyphenyl)piperazine, N-pyrimidin-2-ylpiperazine, cycloheptanylamine, p-trifluorobenzylamine, benzylamine, 3-inidazol-1-ylpropylamine, exo-norboranyl-2-amine, N-phenylethylenediamine, 1-methylbenzylamine, 3,4-(1,3-dioxolanyl)benzylamine, pyridin-2-ylmethylamine, pyridin-3-ylmethylamine, pyridin-4-ylmethylamine, thiophen-2-ylmethylamine, 3,3-dimethylbutylamine, o-methoxybenzylamine, 1-(3-aminopropyl)pyrrolidin-2-one, N-methylethylenediamine, m-methylbenzylamine, 3-methylbutylamine, heptylamine, 3-butoxypropyamine, 3-isopropoxypropylamine, 2-morpholin-4-ylpropylamine, Nl,Nl-diethylethylenediamime, 2-ethylthioethylamine, 4-(2-aminoethyl)phenol, furfurylamine, 4-aminomethylpiperidine, 2-(2-aminoethyl)pyridine, 2-phenoxyethylamine, 2-thiopheneethylamine, p-methoxybenzylamine, 2-(N,N-dimethylamino)ethylamine, 1-amino-2-propanol, 5-methylfurfurylamine, 3-(dimethylamino)propylamine, o-methoxybenzylamine, 1-(3-aminopropyl)-2-pipecoline, hydrazine, and 4-hydroxypiperidine.

### 6,8-disubstituted Adenosine Library

To a solution of commercially available 8-bromoadenosine (100 mg) in 5.0 ml of DMF was added phenylacetylene (1.45 mmol, 148 mg), triphenylphosphine (1.57 mg), copper(I)Diodide (0.38 mg), tris(dibenzylideneacetone) palladium (6.2 mg) and 1.0 ml of triethylamine. The mixture was heated at 100° C. for overnight under argon with vigorous stirring. The hot reaction mixture was filtered through FW14 celite, washed with hot 1, 4-dioxane, and the solvent was evaporated. The residue was purified by column chromatography on silica gel eluted with ethyl acetate/hexane (5: 1) to give the intermediate 35 (supra) (R¹=phenylacetylenyl) in 75% yield.

To a solution of intermediate 35 (R¹=H, 20.0 g) in 300 ml of pyridine was added tetrabutyldimethylsilyl chloride (1.2 eq., 13.53 g). The mixture was stirred overnight at room temperature. The reaction mixture was evaporated and the residue was purified by column chromatography on silica gel eluted with 10% methanol in dichloromethane to give the intermediate 36 (R¹=H) in 100% yield.

A solution of intermediate 36 (R¹=H, 74.6 mmol) in 300 ml of pyridine was added 4.5 eq. of acetic anhydride (34.4 g) under argon. The mixture was stirred at room temperature for 24 hours. The reaction was quenched by adding 40 ml of methanol and 10 ml of water. The mixture was evaporated partially. The residue was extracted by ethyl acetate and diethyl ether. The combined extracts were washed with brine and dried over anhydrous magnesium sulfate. After solvent evaporation, the residue was purified by column chromatography on silica gel eluted with 10% methanol in dichloromethane to give the intermediate 37 (R¹=H) in 89% yield.

A solution of intermediate 37 (R¹=H, 16.0 g) in 25 ml of THF was added to 47.3 ml of 1.0 M tetrabutylammonium fluoride in THF, which was adjusted to pH 6.5 by adding acetic acid. The mixture was stirred at room temperature for 5 hours. After solvent evaporation, the residue was purified by column chromatography on silica gel eluted with 5% methanol in dichloromethane to give the intermediate 38 (R¹=H) in 100% yield.

To a suspension of 4-methoxytrityl chloride resin (1.73 mmol/g, 500 mg) in 1.0 ml of dichloromethane was added 4.0 ml of pyridine and 690 mg of intermediate 38. The reaction mixture was shaken at room temperature for 15 hours. The reaction was quenched by adding 1.0 ml of methanol. The resin was filtered and washed three times with N,N-dimethylformamide, three times with methanol and three times with dichloromethane. After drying on vacuum overnight the intermediate resin 39 (R¹=H) was given in 86% yield.

To the intermediate resin 39 (300 mg) in 1.0 ml of THF was added triphenylphosphine (3.0 eq., 218 mg, 0.83 mmol) and 4-nitrophenethyl alcohol (3.0 eq., 138 mg, 0.83 mmol) in 2.0 ml of THF under argon. The mixture was shaken for 2 min. and then added 0.13 ml of diethyl azodicarboxylate (3.0 eq., 0.83 mmol) at 0° C. The mixture was shaken continuously overnight at room temperature under argon. The resin was filtered and washed three times with THF, three times with methanol and three times with dichloromethane. After drying at room temperature for 3 hours the intermediate resin 40 (R¹=H, R²=4-nitrophenylethyl) was given in 89% yield. 9.0 eq. of alcohol and reagents was used in adenosine library production on Quest 210 Parallel Synthesizer and Advanced ChemTech Vanguard 48 and 96 well Parallel Synthesizer. 48 alcohols were used as building blocks in this step.

3.5 ml of 2.0 M methylamine in methanol was added to each well which contains ˜100 mg of resin 40 (substitution 1.0 mmol/g) on the Vanguard 48 reaction block. The reaction block was shaken at room temperature for three hours. Then the resin was filtered and washed as above (three times with N,N-dimethylformamide, three times with methanol and three times with dichloromethane) to give the intermediate resin 41.

The intermediate resin 41 in 96 reaction vessels were treated with 1.5 ml of 1.5% trifluoroacetic acid in dichloroethane at room temperature for 3 min. The cleaved solution containing adenosine products were collected in 96 glass vials on a cleavage block. To each vial was added 0.2 ml of toluene and evaporated on SpeedVac at room temperature. The final products 42 were given in 20-30 mg and analyzed by TLC and LC-MS spectrometry. The results of LC-MS analysis confirmed the integrity and purity of the compounds. The average purity of the adenosine library is 50-60%.

The following alkyl, aryl and alkynyl reagents (R₁ building blocks) were used for this library: phenylacetylenyl, isopropylacetylenyl, n-butylacetylenyl, tetrabutylphenylacetylenyl, phenylethylacetylenyl, 3-cyclohexylpropyl, 5-cyanopentynyl, 4-tetrabutylphenylethyl, 3-methylbutyl, 5-cyanopentyl, methyl, phenylmethylacetylenyl, phenyl, hydrogen, 2-phenylethyl, 4-methylphenyl, 4-fluorophenyl, 1-hexyl, thiomethoxy, N-methylcarbonyl, 4-biphenyl, N-ethylcarbonyl, 2-thiophenyl and 2-furanyl.

The following alcohol reagents (R₂ building blocks) were used for this library: 1-butanol, 4-nitrophenethyl alcohol, 4-chlorobenzyl alcohol, 1-propanol, 4-nitrobenzyl alcohol, 4-methylbenzyl alcohol, 2-butanol, benzyl alcohol, 2-methyl-1-propanol, crotyl alcohol, 2-norbomanemethanol, 2-methylcyclopropane-methanol, 3-buten-1-ol, neopentyl alcohol, cyclohexylmethanol, 4-trifluorobenzyl alcohol, 3-methyl-2-butem-1-ol, cyclopentanemethanol, 3-methyl-3-buten-1-ol, 4-methyl-1-pentanol, 3-chlorobenzyl alcohol, 3-cyclohexane-1-methanol, 3,3-dimethylbutanol, 3-trifluorobenzyl alcohol, cinnamyl alcohol, tetrahydrofurfuryl alcohol, ethanol, cyclopropyl alcohol, 1-methyl-3-piperidinemethanol, decahydro-2-naphthol, 9-decen-1-ol, 3-cyclopentyl-1-propanol, 1-methyl-2-pyrrolidineethanol, 3-methylbenzyl alcohol, 3-fluorobenzyl alcohol, 3-phenoxybenzyl alcohol, 4-isopropylbenzyl alcohol, 4-methoxybenzyl alcohol, 3,4-dimethoxybenzyl alcohol, 3,5-dimethylbenzyl alcohol, 4-benzyloxybenzyl alcohol, 2-phenylethanol, 4-fluorobenzyl alcohol, phenoxyethanol, benzyloxyethanol, 1-pentanol and 3-pentanol.

Thus, specific embodiments and applications of methods for generating nucleoside libraries have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced.

1. A method of generating a nucleoside library, comprising:
providing a first nucleoside and a second nucleoside, each having a first reactive group protected with a first protecting group and a second reactive group protected with a second protecting group, wherein the first and second nucleosides are coupled to a solid support;
removing the first protecting group from the first and second nucleosides and reacting the first reactive group of the first nucleoside with first reagent and the first reactive group of the second nucleoside with a second reagent; and
removing the second protection group from the first and second nucleosides and reacting the second reactive group of the first nucleoside with a third reagent and the second reactive group of the second nucleoside with a fourth reagent.

2. The method of claim 1 wherein the first and second reagents are chemically identical.

3. The method of claim 1 wherein the step of reacting the second reactive group of the first nucleoside with the third reagent and the second reactive group of the second nucleoside with the fourth reagent is performed in separate compartments.

4. The method of claim 1 wherein at least one of the first and second nucleosides is coupled to a solid support via a linker molecule.

5. The method of claim 4 wherein the coupling of the linker molecule to the nucleoside comprises an acetal bond.

5. The method of claim 1 wherein the first nucleoside and the second nucleoside are chemically distinct from each other.

6. The method of claim 1 wherein at least one of the first and second nucleosides comprises a ribofuranose and a natural nucleoside base.

7. The method of claim 1 wherein the solid support comprises a polystyrene resin or a polystyrene-polyethylene glycol copolymer.

8. The method of claim 1 wherein the first protecting group comprises ap-methoxy-benzyl group and the second protecting group comprises a benzyl group.

9. The method of claim 1 wherein at least one of the first, second, third, and fourth reagent comprises a nucleophile.

10. The method of claim 9 wherein at least one of the first, second, third, and fourth reagents are selected from the group consisting of a Grignard reagent, an organo-lithium reagent, and a Wittig reagent.

11. A method of generating a nucleoside library, comprising:
providing a first sugar and a second sugar, wherein each of the first and second sugars have at least one reactive group and are covalently bound to a solid support;
reacting the first and second reactive groups with a first heterocyclic base and a second heterocyclic base, respectively, thereby forming a first nucleoside and a second nucleoside; and
reacting the first and second nucleoside with a first and second reagent in at least one subsequent reaction, respectively, thereby forming a first modified nucleoside and a second modified nucleoside.

12. The method of claim 11 wherein the first and second sugars are chemically identical.

13. The method of claim 11 wherein the step of reacting the first and second nucleosides with a first and second reagent, respectively, is performed in separate compartments.

14. The method of claim 11 wherein at least one of the first and second sugars is coupled to a solid support via a linker molecule.

15. The method of claim 11 wherein the coupling of the linker molecule to the nucleoside comprises an acetal bond.

16. The method of claim 11 wherein the first and second heterocyclic bases are chemically distinct from each other.

17. The method of claim 11 wherein the heterocyclic base comprises a moiety selected from the group consisting of an imidazole, a thiazole, and an oxazole.

18. The method of claim 11 wherein at least one of the first and second sugars comprises a ribofuranose.

19. The method of claim 11 wherein the solid support comprises a polystyrene resin or a polystyrene-polyethylene glycol copolymer.

20. The method of claim 11 wherein the first and second modified nucleosides are formed in two subsequent reactions.

21. A method of generating a nucleoside library, comprising:
providing a first nucleoside and a second nucleoside, each having a reactive group and each being coupled to a solid support; and
reacting the reactive group of the first and second nucleoside with a first reagent and second reagent, respectively, thereby forming a first modified nucleoside and a second modified nucleoside, wherein the second modified nucleoside is chemically distinct from the first modified nucleoside.

22. The method of claim 21 wherein at least one of the first and second nucleosides comprises a purine heterocyclic base.

23. The method of claim 21 wherein each of the first and second nucleosides comprises a sugar moiety and a heterocyclic base, and wherein the reactive group is disposed on the heterocyclic base of the first and second nucleosides.

24. The method of claim 21 wherein each of the first and second nucleosides further comprises a second reactive group.

25. The method of claim 24 further comprising a step of reacting the second reactive group of the first and second nucleoside with a third reagent and a fourth reagent, respectively.

26. The method of claim 21 wherein the first and the second reagents are selected from the group consisting of an alkyl, an aryl, an alkynyl, an alcohol, and an amine.
